(19) United States
(12) Patent Application Publication
(10) Pub. No.: US 2026/0091007 A1
(43) Pub. Date: Apr. 2, 2026

(54) PHARMACEUTICAL TOPICAL FORMULATIONS OF LIDOCAINE AND KETAMINE

(71) Applicants: Amol Soin, Dayton, OH (US); Aviraj Gabriel Soin, Dayton, OH (US); Dhilen Noah Soin, Dayton, OH (US); Brijen David Soin, Dayton, OH (US); Md Nafiujjaman, San Diego, CA (US); Andrew Xian Chen, San Diego, CA (US)

(72) Inventors: Amol Soin, Dayton, OH (US); Aviraj Gabriel Soin, Dayton, OH (US); Dhilen Noah Soin, Dayton, OH (US); Brijen David Soin, Dayton, OH (US); Md Nafiujjaman, San Diego, CA (US); Andrew Xian Chen, San Diego, CA (US)

(21) Appl. No.: 19/343,258

(22) Filed: Sep. 29, 2025

Related U.S. Application Data
(60) Provisional application No. 63/701,619, filed on Oct. 1, 2024.

(51) Int. Cl. A61K 31/167; A61K 9/00; A61K 9/06; A61K 31/135; A61K 47/06; A61K 47/10; A61K 47/12; A61K 47/14; A61K 47/18; A61K 47/20; A61K 47/22; A61K 47/26; A61K 47/32

(52) U.S. Cl. CPC A61K 31/167; A61K 9/0014; A61K 9/06; A61K 31/135; A61K 47/06; A61K 47/10; A61K 47/12; A61K 47/14; A61K 47/18; A61K 47/20; A61K 47/22; A61K 47/26; A61K 47/32

(57) ABSTRACT

The present invention provides a topical formulation comprising active ingredient(s) lidocaine, ketamine, or pharmaceutically acceptable salt thereof for dermal application and method of use to treat or relieve pain. The topical formulation comprises active pharmaceutical agents lidocaine, ketamine, or their pharmaceutical salt thereof; one or more skin penetration enhancers; and a drug delivery matrix system. The topical formulations are aimed at relieving pain including any type of pain in body, joint pain, chronic pain states such as post-herpetic neuralgia, complex regional pain syndrome, and arthritis. The topical formulation dries and forms a clear film on the skin and as it comprises water insoluble excipients, it is reasonably wash resistant.

### TECHNICAL FIELD

The present invention generally relates to a topical pharmaceutical formulation. Specifically, the present invention relates to a topical formulation comprising active pharmaceutical agents lidocaine and ketamine for relieving pain.

### BACKGROUND

Background description includes information that may be useful in understanding the present invention. It is not an admission that any of the information provided herein is prior art or relevant to the presently claimed invention, or that any publication specifically or implicitly referenced is prior art.

One of the challenging areas where delivery of the drug is crucial is pain management. Chronic pain is a persistent pain that lasts for weeks, months, or even years. It can arise from an initial injury, such as a back sprain, or there might be an ongoing cause, such as illness. However, in some cases, there is no clear cause. Chronic pain can significantly affect an individual's quality of life, leading to difficulties in work, social life, and overall mental health. Effective pain management often requires a multidisciplinary approach involving healthcare professionals from various fields. Traditional routes of administration such as oral or injectable routes have proven to be not very effective. Patients, particularly, for elder and paediatric patients with needle phobia or those requiring long-term administration of medicines such traditional routes are not preferred. Also to reduce the risk of infections associated with injectable therapies, non-invasive approaches are desirable. Accordingly, because the limited efficacy of existing therapies and challenges, there has been intense interest in developing new therapies and mode of delivery.

Transdermal delivery of drugs represents an innovative and increasingly popular method for drug delivery for pain management. Topical preparations like ointments, plasters, and patches are developed to achieve systemic or localized therapeutic effects, offer a non-invasive and painless alternative to injections, reduce side effects, and improve patient compliance. However, such topical preparations are known to provide only immediate delivery and hence are not effective for a longer duration pain management. Further, the currently available plasters and patches are of specific size and shape, For example marketed plasters and patches are clearly visible on the patient wearing the same. This could be undesirable if it is in an area that people can see like the face, jaws, neck, or extremities. Besides, such plasters and patches cannot be applied to difficult to reach areas, corners, inside or on the joints. In addition, some patients have very sensitive skin even to touch or in case of post-herpetic neuralgia after shingles or complex regional pain syndrome, there is a lot of cutaneous pain or some cases of sunburns are very painful. For such painful areas plasters and patches do not offer remedy, especially for cutaneous pain, on the contrary, they can aggravate the pain. In addition, current treatments require topical application that can numb the pain and a separate oral medication to manage systemic pain, where only a small percentage of drug can reach the extremity in pain thereby rendering such treatment modes inefficient. Also, plasters, patches become wet and can pose risk of infection.

There is, therefore, a need in the art to provide a topical formulation for transdermal delivery to relieve pain, especially topical formulation, which is colourless and clear when applies, can be applied easily on a desired area, suitable for regional pain that can undergo desensitization with physical therapy and providing sustained release of the active agent.

### BRIEF SUMMARY

Accordingly, in an aspect, the present invention provides a topical formulation for relieving pain, in particular a topical formulation which is colorless and clear when applied, can be applied easily on a desired area, is able to sustain getting wet within reason so that it does not wash away with water or sweat.

In an aspect, the present invention provides a topical formulation comprising combination of active pharmaceutical agents are lidocaine and ketamine; and water insoluble pharmaceutically acceptable excipient(s), particularly delivery matrix system that can first allow to numb the extremity and provide physical therapy and more desensitization to help into remission and cutaneous pain relief in sustained manner.

In one aspect, the present invention provides a topical formulation comprising:
(a) active pharmaceutical agents are lidocaine, and ketamine, or pharmaceutically acceptable salt thereof;
(b) one or more skin penetration enhancers; and
(c) a drug delivery matrix system.

In yet another aspect, the present invention provides a single dose or multiple dose packaged topical formulation comprising:
(a) active pharmaceutical agents are lidocaine, and ketamine, or pharmaceutically acceptable salt thereof;
(b) one or more skin penetration enhancers; and
(c) a drug delivery matrix system.

In one more aspect, the present invention relates to a method of treating or relieving pain comprising topically administering an effective amount of a topical formulation to a site in need thereof, wherein the formulation comprises:
(a) active pharmaceutical agents are lidocaine, and ketamine, or pharmaceutically acceptable salts thereof;
(b) one or more skin penetration enhancers selected from the group consisting of diethylene glycol monoethyl ether, polyoxyethylene 20 sorbitan monolaurate, ethyl oleate, 1,8-cineole, linalool, L-menthol, dimethyl sulfoxide (DMSO), oleic acid, and polyoxyethylene 20 sorbitan monooleate; and
(c) a drug delivery matrix system comprising methacrylic acid-ethyl acrylate copolymer, triethanolamine, isopropyl myristate, glycerol, triethyl citrate, alcohol anhydrous, and distilled water; and
wherein the formulation releases the active pharmaceutical agents are lidocaine, and ketamine, or pharmaceutically acceptable salts thereof from 0 to 24 hours after administration of the formulation to the site.

Various objectives, features, aspects, and advantages of the inventive subject matter will become more apparent from the following detailed description of preferred embodiments.

### BRIEF DESCRIPTION OF THE FIGURES

Other features of the invention will become more evident from a consideration of the following brief descriptions of drawings:

FIG. 1 is a schematic diagram of a drug delivery matrix system comprising methacrylic acid-ethyl acrylate copolymer, triethanolamine, isopropyl myristate, glycerol, triethyl citrate, ethanol, and water, configured to incorporate and release lidocaine and ketamine.

FIG. 2 is a cross-sectional schematic of skin illustrating topical application of the formulation, the formation of a film barrier, and the controlled diffusion of lidocaine and ketamine into epidermal and dermal tissue.

FIG. 3 is a graph showing percent permeation of lidocaine over a 24-hour period for representative formulations F1, F3, and F7-F12.

FIG. 4 is a graph showing percent permeation of ketamine hydrochloride over a 24-hour period for representative formulations F1, F3, and F7-F12.

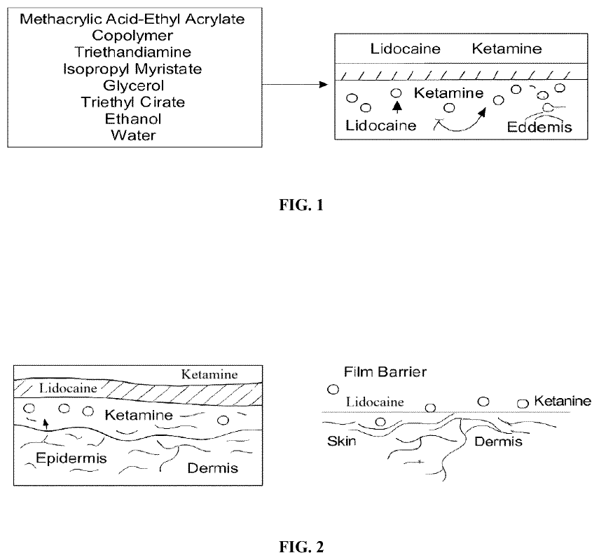

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

The description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein. It must be noted that as used herein and in the appended claims, the singular forms “a,” “an,” and “the” include plural references unless the context clearly dictates otherwise.

Wherever a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both limits, ranges excluding either or both of those included limits are also included in the invention.

The compositions disclosed herein can be delivered by transdermally, by a topical route, formulated as applicator sticks, solutions, suspensions, emulsions, gels, creams, ointments, pastes, jellies, paints, powders, and aerosols.

Pharmaceutical compositions provided herein include compositions wherein the active ingredients (APIs) or drugs are contained in a therapeutically effective amount, i.e., in an amount effective to achieve its intended purpose. The actual amount effective for a particular application will depend, inter alia, on the condition being treated. When administered in methods to treat a disease/injury, such compositions will contain an amount of active ingredient effective to achieve the desired result, e.g., reducing, eliminating, or slowing the progression of pain or inflammation e.g. chronic pain. Determination of a therapeutically effective amount of API or drug disclosed herein is well within the capabilities of those skilled in the art, especially in light of the detailed disclosure herein. The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease/injury being treated (e.g. pain, inflammation, etc.), kind of concurrent treatment, complications from the disease being treated or other health-related problems.

Other therapeutic regimens or agents can be used in conjunction with APIs/drugs disclosed herein. In some embodiments, co-administration can be accomplished by co-formulation, i.e., preparing a single pharmaceutical composition including both active agents. In other embodiments, the active agents can be formulated separately. In another embodiment, the active and/or adjunctive agents may be linked or conjugated to one another.

In embodiments, the compositions described herein may be combined with other pain-relieving agents.

The term “about” in the context of a numerical value means the nominal numerical value±10% thereof, unless expressly indicated otherwise.

A “pharmaceutically acceptable excipient or carrier” as used herein refers to a vehicle capable of suspending or dissolving the active compound, and having the properties of being substantially nontoxic and non-inflammatory in a patient. A pharmaceutically acceptable carrier may be a liquid or cream and, desirably, is suitable for topical application, e.g., application to the skin. As such, the term “pharmaceutically acceptable carrier” encompasses carrier materials approved for use in topical cosmetics. Moreover, a pharmaceutically acceptable carrier may include a pharmaceutically acceptable additive, such as a preservative, antioxidant, fragrance, emulsifier, dye, or excipient known or used in the field of drug formulation and that does not significantly interfere with the therapeutic effectiveness of the biological activity of the active agent, and that is non-toxic to the patient.

The term “excipient” is used herein to describe any ingredient other than an active agents described herein.

As used herein, the term “therapeutically effective amount” refers to an amount of an active compound that, when administered to a patient/mammal, reduces or eliminates pain.

“Topical composition or administration” as used herein refers to the application of a pharmaceutically acceptable formulation to the external surface of a patient, such that the active agents enters the underlying tissue. The external surface may be the skin and topical administration may involve application of a pharmaceutically acceptable formulation to intact, broken, or raw skin or to an open skin wound.

“Transdermal administration” or “transdermally administering” as used herein refers to the diffusion of an agent across the barrier of the skin resulting from topical administration or other application of a pharmaceutically acceptable formulation.

The topical formulations useful in the subject invention can be made into a wide variety of product types. These include, but are not limited to, lotions, creams, gels, sticks, sprays, ointments, pastes, mousses, and cosmetics. The product types can include several types of carrier systems including, but not limited to solutions, emulsions, gels, solids, and liposomes. Techniques for formulation and administration are standard in the art and can be found, for example, in Remington: The Science and Practice of Pharmacy, 20 edition; Lippincott Williams & Wilkins, Philadelphia, PA. Eds Gennaro A. R. et al, 2000 (hereafter “Remington”, the entire disclosure of which is hereby incorporated by reference). The formulation can be selected to maximize delivery to a desired target site in the body such as the skin. As is appreciated by the ordinarily skilled artisan, the specific base to be used is one that provides for optimum delivery for the active agent/drug chosen for a given formulation, and, preferably, provides for other desired characteristics as well, e.g., emolliency or the like.

Lotions, which are preparations that are to be applied to the skin surface, are typically liquid or semi-liquid. Lotions may be formulated with an aqueous or oily base and will in general also contain one or more emulsifying agents, stabilizing agents, dispersing agents, suspending agents, thickening agents, preservatives or coloring agents.

Creams containing the active agent for delivery according to the present invention are viscous liquid or semisolid emulsions, usually oil-in-water or water-in-oil. Cream bases generally contain an oil phase, an emulsifier and an aqueous phase. The oil phase usually, although not necessarily, contains ingredients such as mineral, animal and vegetable oils and waxes, fatty acids, alcohols, amides and esters; the aqueous phase usually, although not necessarily, exceeds the oil phase in volume, and generally contains ingredients such as alcohols, polyols, humectants, viscosity builders and preservatives. The emulsifier in a cream formulation, as described in Remington, is generally a non-ionic, anionic, cationic or amphoteric surfactant.

Gel formulations can also be used in connection with the present invention. As is appreciated by those working in the field of topical drug formulation, gels are viscous semisolid systems. Gels contain organic macromolecules distributed substantially uniformly throughout the carrier liquid, which is typically aqueous, but also may contain solvents, co-solvents and other pharmaceutically known and acceptable adjuvants.

Ointments, which are semisolid preparations, are typically based on petrolatum or other petroleum derivatives. As with other carriers or vehicles, an ointment base should be inert, stable, non-irritating and non-sensitizing. As described, for example, in Remington at pages 845-849, ointment bases may be grouped in four classes: oleaginous bases, absorption bases, water-removable bases, and water-soluble bases. Oleaginous ointment bases include, for example, vegetable oils, fats obtained from animals, and semisolid hydrocarbons obtained from petroleum. Absorption bases, also known as emulsifiable ointment bases, contain little or no water and include, for example, hydroxystearin sulfate, anhydrous lanolin and hydrophilic petrolatum. Absorption bases are generally water-in-oil (W/O) emulsions and include, for example, cetyl alcohol, glyceryl monostearate, lanolin and stearic acid. Water-soluble ointment bases can be prepared from polyethylene glycols of varying molecular weight.

Useful formulations of the invention also encompass sprays. Sprays generally provide the active agent in an aqueous and/or alcoholic solution which can be misted onto the skin for delivery. Such sprays include those formulated to provide for concentration of the active agents solution at the site of administration following delivery, e.g., the spray solution can be primarily composed of alcohol or other like volatile liquid in which the drug or active agent can be dissolved. Upon delivery to the skin, the carrier evaporates, leaving concentrated active agent at the site of administration.

A topical formulation for use in the present invention may also include suitable solid or gel phase carriers for modifying viscosity. Examples of such carriers include, but are not limited to, calcium carbonate, calcium phosphate, various sugars, starches, cellulose derivatives, gelatin, and polymers such as polyethylene glycols.

Further, a topical formulation may include a suitable emulsifier, i.e., an agent that enhances or facilitates mixing and suspending oil-in-water or water-in-oil. An emulsifying agent for use in the invention may consist of a single emulsifying agent or may be a blend of emulsifying agents and may be a nonionic, anionic or cationic surfactant or a blend of two or more such surfactants. Such surface-active agents are described, for example, in “McCutcheon's Detergent and Emulsifiers”, North American Edition, 1980 Annual published by the McCutcheon Division, MC Publishing Company, 175 Rock Road, Glen Rock, NJ. 07452, USA. Especially suitable non-ionic emulsifying agents for inclusion in the pharmaceutically acceptable formulations for use in the present invention are those with a hydrophile-lipophile balance (HLB) as determined by the method described, for example, by Paul L. Lindner in “Emulsions and Emulsion”, edited by Kenneth Lissant, published by Dekker, New York, N. Y., 1974, pages L88-190. Examples of such nonionic emulsifiers include, but are not limited to, “BRIJ 72”, the trade name for a polyoxyethylene (2) stearyl ether having an HLB of 4.9; “BRIJ 721”, the trade name for a polyoxyethylene (21) stearyl ether having an HLB of 15.5.

A topical pharmaceutical formulation may also contain suitable emollients. Emollients are materials that may be used for the prevention or relief of dryness, as well as for the protection of the skin. Useful emollients include, but are not limited to, cetyl alcohol, isopropyl myristate, stearyl alcohol, and the like. A wide variety of suitable emollients are known in the art and can be used in the formulations encompassed by the invention. See e.g., Sagarin, Cosmetics, Science and Technology, 2nd Edition, Vol. 1, pp. 32-43 (1972), and U.S. Pat. No. 4,919,934, to Deckner et al., both of which are incorporated herein by reference in their entirety.

Topical formulations of the present invention may further contain suitable chelating agents to form complexes with metal cations which do not cross a lipid bilayer. Examples of suitable chelating agents include ethylene diamine tetraacetic acid (EDTA), ethylene glycol-bis (beta-aminocthylether-N,N,N-tetraacetic acid (EGTA) and 8-amino-2-[(2-amino-5-methylphenoxy) methyl]-6-methoxyquinoline-N,N,N′-5N′-tetraacetic acid, tetrapotassium salt (QUIN-2).

Topical formulations of the invention may also include suitable neutralizing or buffering agents used to adjust the pH of the formulation to optimize the delivery of the drug.

A topical pharmaceutical formulation may also contain one or more suitable solvents and cosolvents. Suitable solvents and cosolvents which are exemplified may include ingredients such as ethanol, propylene glycol, glycerin, dipropylene glycol and polyethylene glycol.] Further to any embodiments disclosed herein, the antioxidant may be added as excipient selected from acetyl cysteine, acetyl trihexylcitrate, arbutin, ascorbic acid, ascorbic acid polypeptide, ascorbyl dipalmitate, ascorbyl methylsilanol pectinate, ascorbyl palmitate, ascorbyl stearate, betalains (betanin), betaxanthine (e.g., indicaxanthinc) BHA, BHT, t-butyl hydroquinone, calcium ascorbate, chitosan salicylate, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, crythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate other monovalent or divalent salts of ascorbyl phosphate, methylsilanol ascorbate, natural botanical anti-oxidants such as green tea or grape seed extracts, nordihydroguaiarctic acid, octyl gallate, phenylthioglycolic acid, potassium ascorbate, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiomorpholinone, thiosalicylic acid, thiotaurine, thioxanthine, tocophereth-5, tocophereth-10, tocophereth-12, tocophereth-18, tocophereth-50, tocopherol, tocophersolan, tocopheryl acetate, tocopheryl linoleate, tocopheryl nicotinate, tocoquinone, tocopheryl succinate, triethyl citrate, tris (nonylphenyl) phosphite, ubiquinone, zinc dibutyldithiocarbamate, ascorbyl-2-phosphate and ascorbyl polyphosphate and the respective salts thereof, allantoin ascorbate, ascorbyl tocopheryl maleate, potassium ascorbyl tocopheryl phosphate, tetrahexyldecyl ascorbate, tetra hydrodiferuloylmethane, caffeic acid, caffeoylquinic acid (from Scheffera Heptphylla) (L.) Frodin) in particular 3-O-caffeoyl quinic acid, melatonin, thiotaurine, ortho tolyl biguanidine, telmesteine, and aconitic acid.

In embodiments, the pharmaceutical composition is within a container that is substantially free of oxygen gas. In embodiments, the container includes an inert gas. In embodiments, the inert gas is nitrogen or argon.

In embodiments, the pharmaceutical composition further includes a pH adjuster. In embodiments, the pH adjuster is an inorganic acid, e.g., HCL, or an inorganic base, e.g., NaOH or KOH. In embodiments, the pH adjuster is prepared in anhydrous organic solvent. In embodiments, the pharmaceutical composition is a topical pharmaceutical composition.

In embodiments, the pharmaceutical composition may further includes a gelling agent, as known in the art. In embodiments, the gelling agent is CARBOPOL® Ultrez 30 (i.e., INCI designation carbomer, cross-linked homopolymer of acrylic acid), CARBOPOL® Ultrez 10 (INCI designation carbomer, cross-linked homopolymer of acrylic acid), CARBOPOL® Ultrez 21 (INCI designation acrylates/C10-30 alkyl acrylate crosspolymer, with hydrophobically modified crosslinked polyacrylate polymer) or mixtures thereof.

In embodiments, the pharmaceutical formulation may further includes a non-aqueous liquid carrier. In embodiments, the non-aqueous liquid carrier is glycerol, sorbitol, mannitol, erythritol, xylitol, glyceryl triacetate, propylene glycol, a polyethylene glycol, and a polymeric polyol. In embodiments, the non-aqueous liquid carrier is glycerol.

Liquid forms, such as lotions suitable for topical administration or suitable for cosmetic application, may include a suitable aqueous or nonaqueous vehicle with buffers, suspending and dispensing agents, thickeners, penetration enhancers, and the like. Solid and semi-solid forms such as creams, ointments, sticks or pastes or the like may include, for example, any of the following ingredients, water, oil, alcohol or grease as a substrate with surfactant, polymers such as polyethylene glycol, thickeners, solids and the like. Liquid or solid formulations may include enhanced delivery technologies such as liposomes, microsomes, microsponges, patches, and the like.

A pharmaceutically acceptable topical formulation may include a pharmaceutically acceptable carrier such as water, oils (including vegetable and mineral oils), cream bases, lotion bases, ointment bases, and the like. These bases include suspending agents, thickeners, penetration enhancers, and the like. Topical and transdermal formulations are well known to those in the art of cosmetics and topical pharmaceuticals and are described, for example, in Chapter 44 of Remington.

Topical (e.g., transdermal) formulations may also include pharmaceutically acceptable vehicles. Additives for topical formulations are well-known in the art, and may be added to the topical composition, as long as they are pharmaceutically acceptable and not deleterious to the epithelial cells or their function. Further, the additives should not cause deterioration in the stability of the formulation, in particular, of the active compound. For example, inert fillers, anti-irritants, tackifiers, excipients, fragrances, opacifiers, antioxidants, gelling agents, stabilizers, surfactants, emollients, coloring agents, preservatives, buffering agents, other permeation enhancers, and other conventional components of transdermal delivery devices as are known in the art. Excipients generally are carriers, diluents and/or vehicles used in formulating drug compositions. Excipients are standard in the art and examples of excipients and their application can be found, for instance, in Ratz, M., Drug Design 4:93-148, 1973.

The term “sustained release (SR)” used herein covers a type of pharmaceutical dosage form that is designed to release the active ingredient (drug) gradually and consistently over an extended period of time, rather than all at once after administration. This controlled release allows for a prolonged therapeutic effect, reducing the frequency with which a patient needs to take the medication, often improving patient compliance and therapeutic outcomes. It covers a gradual drug release, where instead of an immediate spike in drug content in the bloodstream, SR formulations maintain a steady release, aiming to keep drug levels within the therapeutic window for longer periods. Further, the medication remains active in the body for extended periods, which is especially beneficial for drugs with a short half-life that would otherwise require frequent dosing. Also, sustained release reduces the risk of fluctuations (peaks and troughs) in drug concentration, minimizing side effects and maximizing effectiveness.

As used herein, the “lidocaine” is a compound having IUPAC name, 2-(diethylamino)-N-(2,6-dimethylphenyl) acetamide and structure as given below.

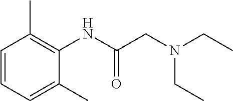 [SMILES: CCN(CC)CC(=O)Nc1c(C)cccc1C]

As used herein, “ketamine” is a compound having IUPAC name, 2-(2-chlorophenyl)-2-(methylamino)cyclohexan-1-one and structure as given below.

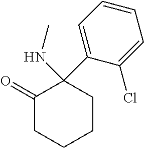 [SMILES: CNC1(c2ccccc2Cl)CCCCC1=O]

The present invention provides a topical formulation comprising active ingredient(s) lidocaine, ketamine, or pharmaceutically acceptable salt thereof for dermal application and method of use to treat or relieve pain. The topical formulation of the present invention is amenable to be applied on an affected area and when applied on the skin, eventually within a few seconds or minutes, dries completely clear on the skin. The topical formulation of the present invention comprises a drug delivery matrix system that carries active pharmaceutical agent(s) that are potentially absorbed through the skin and provide pain relief to the patient.

A topical formulation of the present invention comprises pharmaceutical agents lidocaine and ketamine, or pharmaceutically acceptable salts thereof; and water insoluble pharmaceutically acceptable excipient(s). The topical formulation of the present invention comprises drug delivery matrix system that can first allow desensitization and then physical therapy and more desensitization to help remission and pain relief in sustained manner.

In an embodiment, the present invention provides a topical formulation comprising:
(a) one or more active pharmaceutical agents selected from a group comprising of local anesthetic agent and analgesic agent, or pharmaceutically acceptable salts thereof;
(b) one or more skin penetration enhancers; and
(c) a drug delivery matrix system;
wherein the drug delivery matrix system comprises combination of acrylic based copolymer, alcoholamine, fatty acid ester, polyol, tricarboxylic acid ester, alcohol and solvent.

In an embodiment, the one or more active pharmaceutical agents comprises at least two pharmaceutical agents.

In an embodiment, the local anesthetic agent is selected from lidocaine, bupivacaine, mepivacaine, procaine, tetracaine, and articaine.

In an embodiment, the analgesic agent is selected from ketamine, diclofenac, ketoprofen, ibuprofen, piroxicam, amitriptyline and doxepin.

In an embodiment, the local anesthetic agent is lidocaine, and the analgesic agent is ketamine.

In an embodiment, the one or more skin penetration enhancers are selected from selected from the group comprising of diethylene glycol monoethyl ether, polyoxyethylene 20 sorbitan monolaurate, ethyl oleate, 1,8-cineole, linalool, limonene, L-menthol, dimethyl sulfoxide (DMSO), oleic acid, polyoxyethylene 20 sorbitan monooleate, camphor, farnesol, eugenol, geraniol, α-pinene oxide, tetrahydrogeraniol, cymene, and sodium glycolate.

In an embodiment, the acrylic based copolymer is selected from polymethacrylate based copolymer, methacrylic acid-ethyl acrylate copolymer, methacrylic acid-methyl methacrylate copolymer, ammonio methacrylate copolymer type A, ammonio methacrylate copolymer type B, acrylates/ethylhexyl acrylate copolymer, and sodium polyacrylate.

In an embodiment, said polymethacrylate based copolymer is Eudragit® L, Eudragit® S, Eudragit® RL, or Eudragit® RS.

In an embodiment, the alcoholamine is selected from ethanolamine, diethanolamine, triethanolamine, isopropanolamine, aminomethyl propanol, and 2-amino-2-methyl-1-propanol.

In an embodiment, the fatty acid ester is selected from isopropyl myristate, isopropyl palmitate, ethyl oleate, butyl stearate, cetyl palmitate, octyl stearate, lauryl lactate, and oleyl oleate.

In an embodiment, the polyol is selected from glycerol, propylene glycol, sorbitol, mannitol, xylitol, butylene glycol, and polyethylene glycol.

In an embodiment, the polyethylene glycol is based on PEG 200 to 6000.

Said PEG 200 to 6000 means polyethylene glycol having molecular weight in the range of 200 to 6000.

In an embodiment, the tricarboxylic acid ester is selected from triethyl citrate, tributyl citrate, acetyl triethyl citrate, acetyl tributyl citrate, trimethyl citrate, and triacetin.

In an embodiment, the alcohol is selected from anhydrous ethanol, isopropanol, n-propanol, benzyl alcohol, butanol, and denatured alcohol.

In an embodiment, the solvent is distilled water.

In an embodiment, the drug delivery matrix system comprises combination of methacrylic acid-ethyl acrylate copolymer, triethanolamine, isopropyl myristate, glycerol, triethyl citrate, alcohol anhydrous, and distilled water.

In an embodiment, the drug delivery matrix system comprises:
(a) the acrylic based copolymer is present in an amount of about 10% to about 20% by weight of the drug delivery matrix system;
(b) the alcoholamine is present in an amount of about 1% to about 2% by weight of the drug delivery matrix system;
(c) the fatty acid ester is present in an amount of about 3% to about 8% by weight of the drug delivery matrix system;
(d) the polyol is present in an amount of about 3% to about 8% by weight of the drug delivery matrix system;
(e) the tricarboxylic acid ester is present in an amount of about 2% to about 6% by weight of the drug delivery matrix system;
(f) the alcohol is present in an amount of about 30% to about 80% by weight of the drug delivery matrix system; and/or
(g) the solvent is present in quantity sufficient.

In an embodiment, the one or more active pharmaceutical agents are present in the topical formulation in an amount of about 1% to about 50% by weight.

In an embodiment, the skin penetration enhancer is present in the topical formulation in an amount of about 0.1% to about 50% by weight.

In an embodiment, the drug delivery matrix system is present in the topical formulation in an amount of about 60% to about 98.9% by weight.

In an embodiment, the anesthetic agent or pharmaceutically acceptable salts thereof is present in an amount of about 2% to about 10% by weight;

In an embodiment, the topical formulation comprises:
(a) the analgesic agent or pharmaceutically acceptable salts thereof is present in an amount of about 5% to about 15% by weight;
(b) the one or more skin penetration enhancers is in an amount of about 0.2% to about 15% by weight; and
(c) the drug delivery matrix system is in an amount of about 70% to about 85% by weight.

In an embodiment, the formulation optionally comprises a pharmaceutically acceptable excipients selected from buffer, chelating agent, fragrance, preservative, emulsifier, suspending agent, and thickening agent.

In an embodiment, the formulation is a sustained release formulation.

In one embodiment, the present invention provides a topical formulation comprising:
(a) active pharmaceutical agents are lidocaine, and ketamine, or pharmaceutically acceptable salt thereof;
(b) one or more skin penetration enhancers; and
(c) a drug delivery matrix system.

In one embodiment, the one or more skin penetration enhancer is selected from but not limited to diethylene glycol monoethyl ether, polyoxyethylene 20 sorbitan monolaurate, ethyl oleate, 1,8-cineole, linalool, L-menthol, dimethyl sulfoxide (DMSO), oleic acid, and polyoxyethylene 20 sorbitan monooleate.

In certain embodiment, the skin penetration enhancers is diethylene glycol monoethyl ether, and polyoxyethylene 20 sorbitan monolaurate.

In one embodiment, the present invention provides a topical formulation comprising:
(a) active pharmaceutical agents are lidocaine, and ketamine, or pharmaceutically acceptable salts thereof;
(b) one or more skin penetration enhancers selected from the group consisting of diethylene glycol monoethyl ether, polyoxyethylene 20 sorbitan monolaurate, ethyl oleate, 1,8-cineole, linalool, L-menthol, dimethyl sulfoxide (DMSO), oleic acid, and polyoxyethylene 20 sorbitan monooleate; and
(c) a drug delivery matrix system.

In an embodiment, the drug delivery matrix system comprises methacrylic acid-ethyl acrylate copolymer, triethanolamine, isopropyl myristate, glycerol, triethyl citrate, alcohol anhydrous, and DI-water.

The drug delivery matrix system can comprise:
(a) methacrylic acid-ethyl acrylate copolymer (1:1) present in an amount of about 10% to about 20% by weight;
(b) triethanolamine present in an amount of about 1% to about 2% by weight;
(c) isopropyl myristate present in an amount of about 3% to about 8% by weight;
(d) glycerol present in an amount of about 3% to about 8% by weight;
(e) triethyl citrate present in an amount of about 2% to about 6% by weight;
(f) alcohol anhydrous present in an amount of about 30% to about 80% by weight; and/or
(g) distilled water present in quantity sufficient.

Specifically, the drug delivery matrix system comprises:
(a) methacrylic acid-ethyl acrylate copolymer (1:1) present in an amount of about 16% by weight;
(b) triethanolamine present in an amount of about 1.2% by weight;
(c) isopropyl myristate present in an amount of about 5% by weight;
(d) glycerol present in an amount of about 5% by weight;
(e) triethyl citrate present in an amount of about 4.2% by weight;
(f) alcohol anhydrous present in an amount of about 50% by weight; and/or
(g) distilled water present in quantity sufficient.

The active pharmaceutical agent lidocaine, ketamine or their salt can be present in the topical formulation in an amount of about 1% to about 50% by weight.

The active pharmaceutical agent lidocaine, ketamine or their salt can be present in the topical formulation in an amount of about 1%, 2%, 3%, 4%, 5%, 6%, 7%, 8%, 9%, 10%, 15%, 20%, 25%, 30%, 35%, 40%, 45%, or 50% by weight.

The active pharmaceutical agent lidocaine, ketamine or their salt can be present in the topical formulation from about 2% to about 20% by weight.

In one embodiment, active pharmaceutical agent lidocaine, ketamine or their salt is present in an amount of about 5% to about 10% by weight.

The skin penetration enhancer can be present in the topical formulation in an amount of about 0.1% to about 50% by weight. The one or more skin penetration enhancers can be present in an amount of about 0.2% about 10% by weight.

The skin penetration enhancer can be present in the topical formulation in an amount of about 0.1%. 0.2%, 0.3%, 0.4%, 0.5%, 0.6%, 0.7%, 0.8%, 0.9%, 1%, 2%, 3%, 4%, 5%, 6%, 7%, 8%, 9%, 10%, 15%, 20%, 25%, 30%, 35%, 40%, 45%, or 50% by weight.

The drug delivery matrix system can be present in the topical formulation in an amount of about 60% to about 98.9% by weight. The drug delivery matrix system can be present in the topical formulation in an amount of about 70% to about 85% by weight.

The drug delivery matrix system can be present in the topical formulation in an amount of about 60%, 65%, 70%, 75%, 80%, 81%, 82%, 83%, 84%, 85%, 86%, 87%, 88%, 89%, 90%, 91%, 92%, 93%, 94%, 95V, 96%, 97%, 98%, or 99% by weight.

In one embodiment, the present invention provides a topical formulation comprising:
(a) an active pharmaceutical agent lidocaine, ketamine or their salt present in an amount of about 1% to about 50% by weight;
(b) one or more skin penetration enhancers present in an amount of about 0.1% to about 50% by weight; and
(c) a drug delivery matrix system present in an amount of about 60% to about 98.9% by weight.

In one embodiment, the present invention provides a topical composition comprising:
(a) an active pharmaceutical agent lidocaine, and ketamine, or their salt present in an amount of about 5% to about 20% by weight;
(b) one or more skin penetration enhancers present in an amount of about 0.2% about 10% by weight; and
(c) a drug delivery matrix system present in an amount of about 70% to about 85% by weight.

In one embodiment of the present invention, the topical formulation comprises:
(d) lidocaine or its salt present in an amount of about 2% to about 10% by weight;
(e) ketamine or its salt present in an amount of about 5% to about 15% by weight;
(f) one or more skin penetration enhancer in an amount of about 0.2% to about 15% by weight; and
(g) a drug delivery matrix system in an amount of about 70% to about 85% by weight.

In yet another embodiment of the present invention, the topical formulation comprises:
(a) lidocaine or its salt present in an amount of about 5% by weight;
(b) ketamine or its salt present in an amount of about 10% w by weight;
(c) one or more skin penetration enhancer in an amount of about 0.2% about 10% w/w of the total composition; and
(d) a drug delivery matrix system in an amount of about 75% to about 85% by weight.

The topical formulation of the present invention can further comprise additional pharmaceutically acceptable excipients selected from buffers, chelating agent, fragrance, preservatives, emulsifiers, suspending agent, thickening agent, or any other suitable excipient.

In certain embodiment, the present invention provides packaged topical formulation.

The topical formulation can be packaged into a multidose package or into a single use package that can dispense single dose to simplify the administration of the topical formulation to a subject.

In an embodiment, the present invention provides a single dose or multiple dose packaged topical formulation comprising:
(a) active pharmaceutical agent(s) lidocaine and ketamine or pharmaceutically acceptable salt thereof;
(b) one or more skin penetration enhancers selected from the group consisting of diethylene glycol monoethyl ether, polyoxyethylene 20 sorbitan monolaurate, ethyl oleate, 1,8-cineole, linalool, L-menthol, dimethyl sulfoxide (DMSO), oleic acid, and polyoxyethylene 20 sorbitan monooleate; and
(c) a drug delivery matrix system comprising methacrylic acid-ethyl acrylate copolymer, triethanolamine, isopropyl myristate, glycerol, triethyl citrate, alcohol anhydrous, and DI-water.

In an embodiment, the present invention provides a single dose or multiple dose packaged topical formulation comprising:
(a) lidocaine or it salt present in an amount of about 2% to about 10% by weight;
(b) ketamine or its salt present in an amount of about 5% to about 15% by weight;
(c) one or more skin penetration enhancer in an amount of about 0.2% to about 15% by weight; and
(d) a drug delivery matrix system in an amount of about 70% to about 85% by weight.

In certain embodiment, the present invention provides an HPLC method for determination of lidocaine, ketamine HCl and impurities in a sample.

The HPLC method is robust and meets with the requirement of linearity, quantification limit, and precision.

In another embodiment, the present invention relates to a method of treatment or relieving pain comprising applying the topical formulation to a patient in need thereof.

In an embodiment, the present invention provides a method of treating or relieving pain comprising administering an effective amount of the topical formulation as disclosed above to a mammal in need thereof, wherein the administration is done by applying the topical formulation to a site having pain; and wherein the formulation releases the one or more active pharmaceutical agents, or pharmaceutically acceptable salts thereof for 24 hours or more after administration of the formulation to the site.

In an embodiment, the pain is selected from body pain, joint pain, pain caused by injury, pain of region where a needle is injected into the skin, and chronic pain.

In an embodiment, the pain caused by injury comprises wounds or burns.

In an embodiment, the chronic pain is selected from post-herpetic neuralgia, complex regional pain syndrome, and arthritis.

In an embodiment, the pain is a complex regional pain syndrome and the topical formulation first allows local anesthetic agent to numb the extremity along with analgesic agent to allow the patient to undergo physical therapy and continue to provide desensitization to help into remission of pain.

In one more embodiment, the present invention relates to a method of treating or relieving pain comprising topically administering an effective amount of a topical formulation to a site in need thereof, wherein the formulation comprises:
(a) active pharmaceutical agents are lidocaine, and ketamine, or pharmaceutically acceptable salts thereof;
(b) one or more skin penetration enhancers selected from the group consisting of diethylene glycol monoethyl ether, polyoxyethylene 20 sorbitan monolaurate, ethyl oleate, 1,8-cineole, linalool, L-menthol, dimethyl sulfoxide (DMSO), oleic acid, and polyoxyethylene 20 sorbitan monooleate; and
(c) a drug delivery matrix system comprising methacrylic acid-ethyl acrylate copolymer, triethanolamine, isopropyl myristate, glycerol, triethyl citrate, alcohol anhydrous, and distilled water; and
wherein the formulation releases the active pharmaceutical agents lidocaine, and ketamine, or pharmaceutically acceptable salts thereof from 0 to 24 hours after administration of the formulation to the site.

In one more embodiment, the present invention relates to a method of treating or relieving pain comprising topically administering an effective amount of a topical formulation to a site in need thereof, wherein the formulation comprises:
(a) lidocaine or it salt present in an amount of about 2% to about 10% by weight;
(b) ketamine or its salt present in an amount of about 5% to about 15% by weight;
(c) one or more skin penetration enhancer in an amount of about 0.2% to about 15% by weight; and
(d) a drug delivery matrix system in an amount of about 70% to about 85% by weight; and
wherein the formulation releases the active pharmaceutical agents lidocaine, and ketamine, or pharmaceutically acceptable salts thereof from 0 to 24 hours after administration of the formulation to the site.

In one more embodiment, the present invention relates to a method of treating or relieving pain comprising topically administering an effective amount of a topical formulation to a site in need thereof, wherein the formulation comprises:
(a) active pharmaceutical agents are lidocaine, and ketamine, or pharmaceutically acceptable salts thereof;
(b) a skin penetration enhancer diethylene glycol monoethyl ether; and
(c) a drug delivery matrix system comprising methacrylic acid-ethyl acrylate copolymer, triethanolamine, isopropyl myristate, glycerol, triethyl citrate, alcohol anhydrous, and distilled water; and
wherein the formulation releases the active pharmaceutical agents are lidocaine, and ketamine, or pharmaceutically acceptable salts thereof from 0 to 24 hours after administration of the formulation to the site.

In one more embodiment, the present invention relates to a method of treating or relieving pain comprising topically administering an effective amount of a topical formulation to a site in need thereof, wherein the formulation comprises:
(a) active pharmaceutical agents are lidocaine, and ketamine, or pharmaceutically acceptable salts thereof;
(b) a skin penetration enhancer polyoxyethylene 20 sorbitan monolaurate; and
(c) a drug delivery matrix system comprising methacrylic acid-ethyl acrylate copolymer, triethanolamine, isopropyl myristate, glycerol, triethyl citrate, alcohol anhydrous, and distilled water; and
wherein the formulation releases the active pharmaceutical agents are lidocaine, and ketamine, or pharmaceutically acceptable salts thereof from 0 to 24 hours after administration of the formulation to the site.

The topical formulation of the present invention is applied to an affected area or and adjacent area of pain. The topical formulation is reapplied as necessary. The dosing is applied to the painful area and subcutaneously in order to effect pain relief. Pain relief is obtained within minutes to hours and lasts for periods of approximately three to six hours to twenty-four hours. The topical formulation is applied such that the dosage of the active agents is sufficient to provide an effective dose in the painful area or immediately adjacent areas

The topical formulation of the present invention may be used for local topical delivery to any location where reduction of pain is required or desirable.

In one embodiment, the topical formulation is used to treat pain, wherein the pain is pain in body, joint pain, pain cause by injuries, such as wounds and burns, region where a needle is injected into the skin for example to start an IV or draw blood or inject medicines into a reservoir implanted under the areas where cosmetic and surgical procedures are done chronic pain states such as post-herpetic neuralgia, complex regional pain syndrome, and arthritis.

The topical formulation of the present invention is a gel and when applied, it dries completely clear. The topical formulation when applied to an affected area creates a protective film. The topical formulation can be spread out an affected area allowing coverage over the exact areas that the patient experiencing pain, unlike a regular topical rigid plasters and patches, which can only cover a single rectangular space. Unlike such conventional products, the topical formulation can be applied around curves, bends, folds, and joints of the skin and delivery drug to allow effective pain management. Also, since it forms a colourless film and not visible like traditional topical preparations, it does not look shabby or attract undue attention, thus does not impact social life, and preserve modesty.

The topical formulation comprises active ingredients lidocaine and ketamine, which are local anaesthetics. Lidocaine can block sodium and potassium transmission, which essentially numbs the nerve by preventing action potential of nerve pain signal transmission. Where has the ketamine blocks NMDA receptors which are important in modulating pain and the disease state of complex regional pain syndrome. Ketamine also blocks the polysynaptic neurons in the spinal cord, which can inhibit excitatory neurotransmission and in the periphery. The Neurons are hyper excitable in complex regional pain syndrome which is responsible for the extensive pain. Ketamine blocks excitatory neurotransmission. Lidocaine provides an additional different nerve blocking affect. Thus, using both lidocaine and ketamine together can help synergistically improve the disease state in the two different ways.

The topical formulation comprising excipients like skin enhancer can allow for the transmission of active pharmaceutical ingredients lidocaine and ketamine through the skin that can be absorbed through the cutaneous nerves and underneath the skin to provide pain relief. This could be helpful in joint pain, arthritis, or the like condition. The medications can be time released and can be released over several hours. For example, when applied, the topical formulation forms a film on a patient's area of pain and may last several hours continuously diffusing pharmaceutical agents are lidocaine and ketamine under the skin for a desired therapeutic effect.

The topical formulation comprises a topical delivery matrix system that can allow timed release that can give continuous numbing effect by the lidocaine and continuous relief from the ketamine over several hours.

The topical formulation of the present invention can provide continuous pain relief from about 3 to about 12 hours through the protective barrier formed by the drug delivery matrix before it fully wears off and degrades as well as full use of the medicines in the formulation.

The topical formulation can help allow for the transmission of active pharmaceutical ingredients through the skin that can be absorbed through the cutaneous nerves and underneath the skin to provide pain relief. This can be helpful to subjects suffering from joint pain, arthritis, or the like condition.

The topical formulation of the present invention can help address complex regional pain syndrome, which is characterized by excessive pain in typically extremities, upper and lower extremities manifested by hypersensitivity to touch, allodynia, pseudo motor and vasomotor changes and exquisite tenderness to light palpation or stimulation for example even wearing a pair of socks is painful. Unlike the conventional oral formulations, which can provide systemic spread and only a small percentage of active agent actually reach the extremity in pain, the topical delivery system of the present invention is uniquely advantageous in such disease as it allows all of the medication's to be absorbed over that part of the extremity.

One of the most important long-term treatments of complex regional pain syndrome undergo desensitization with physical therapy by touching, manipulating and moving the extremity. Oftentimes this is impossible because the patient is syndrome is being able to in too much pain, hence, applying the topical formulation of the present invention comprising synergistic combination of lidocaine and ketamine along with a topical drug delivery matrix, can first allow lidocaine to numb the extremity along with ketamine, which can allow the patient to undergo physical therapy and continue to provide desensitization to help into remission of pain.

The topical formulation of the present invention comprises excipients that are water insoluble so that the film formed upon applying the topical formulation can sustain getting wet within reason and it does not wash away with water or sweat.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

### Example 1

### HPLC Method for Determination of Lidocaine, Ketamine HCl and Impurities

For determination of lidocaine, ketamine HCl and impurities in a sample, the following HPLC method was developed by doing linearity, quantification limit, and precision tests.

### Conditions for HPLC Assays:

TABLE 1

| System | HP Agilent 1100; system 30 | | | |
|---|---|---|---|---|
| Column | HALO 90Å, Phenyl-Hexyl (2.7 μm × 4.6 × 150 mm) | | | |
| Mobile phase A | SPB:ACN (9:1) | | | |
| Mobile phase B | 100% | | | |
| | Time | % Mobile | % Mobile | Flow Rate |
| | (Minutes) | Phase A | Phase B | (mL/min) |
| Gradient | 0 | 60 | 40 | 1 |
| | 1 | 60 | 40 | 1 |
| | 10 | 20 | 80 | 1 |
| | 11 | 0 | 100 | 1.5 |
| | 15 | 0 | 100 | 1.5 |
| | 16 | 60 | 40 | 1 |
| | 23 | 60 | 40 | 1 |
| Run Time | 23 minutes | | | |
| Autosampler | 5° C. | | | |
| Temperature | | | | |

| | |
|---|---|
| Column Temperature | 40° C. |
| Detection | UV 210 nm, 254 nm, 280 nm, 220 nm |
| Injection volume | 50 μL |
| Standard Conc. | 0.001 mg/mL |
| Diluent | SPB |

### A. Mobile Phase Preparation:

a. Preparation of diluent (20 mM Sodium Phosphate (SBP) Buffer (pH 7.4)):
1. 800 mL of distilled water was taken in a suitable container.
2. 4043.0 mg of sodium phosphate dibasic heptahydrate (Na₂HPO₄·7H₂O) was added to the distilled water and dissolved.
3. 678.7 mg of sodium phosphate monobasic monohydrate (NaH₂PO₄·H₂O) was added to the solution.
4. Final desired pH was adjusted using HCl or NaOH.
5. Distilled water was added until the volume was 1 L.
b. Mobile Phase A: ACN:SPB=1:9 (V:V)
c. Mobile Phase B: 100% ACN
   B. Lidocaine Standard Stock Solution (0.1 mg/mL):

5 mg of lidocaine was accurately weighed and transferred into a 50-mL of volumetric flask. Weight of 7.7 mg was recorded. To it, diluent (SPB) was added upto approximately ¾ volume of the flask and dissolved with the aid of a sonicator. Final volume was diluted to 1 L by adding dilute and mixed well.

C. Lidocaine Working Solution (0.001 mg/mL):

1 mL of lidocaine standard stock solution was accurately transferred into a 100 mL of volumetric flask. To it, diluent (SPB) was added up to approximately ¾ volume of the flask and dissolved with the aid of a sonicator. Final volume was diluted to 1 L by adding dilute and mixed well.

D. Ketamine HCl Standard Stock Solution (0.1 mg/mL):

5 mg of ketamine HCl was accurately weighed and transferred into a 50-mL of volumetric flask. Weight of 7.5 mg was recorded. To it, diluent (SPB) was added up to approximately ¾ volume of the flask and dissolved with the aid of a sonicator. Final volume was diluted to 1 L by adding dilute and mixed well.

E. Ketamine Working Solution (0.001 mg/mL)

1 mL of ketamine HCl standard stock solution was accurately transferred into a 100 mL of volumetric flask. To it, diluent (SPB) was added up to approximately ¾ volume of the flask and dissolved with the aid of a sonicator. Final volume was diluted to 1 L by adding dilute and mixed well.

### Results:

TABLE 2.1
Precision for Lidocaine

| Sample | RT (min) | Peak Area |
|---|---|---|
| 1 | 4.76 | 316.2 |
| 2 | 4.80 | 315.8 |
| 3 | 4.80 | 315.9 |
| 4 | 4.77 | 315.8 |
| AVG | 4.78 | 315.9 |
| SD | 0.02 | 0.2 |
| % RSD | 0.47 | 0.1 |

AVG: Average
SD: Standard deviation
RSD: Relative standard deviation

### Acceptance Criteria: RSD: ≤2.0%

The peak area relative standard deviation RSD from the four consecutive standard solution injections was 0.1%. The acceptance criteria was thus met.

TABLE 2.2
Linearity for Lidocaine

| Conc. (μg/mL) | Peak Area |
|---|---|
| 0.308 | 62.1 |
| 0.616 | 125.3 |
| 0.924 | 188.7 |
| 1.232 | 251.5 |
| 1.540 | 316.2 |

### Acceptance Criteria: R² Value ≥0.995

The R² value for the five concentration injections was 1. The acceptance criteria was thus met.

TABLE 2.3
Precision for Ketamine HCl

| Sample | RT (min) | Peak Area |
|---|---|---|
| 1 | 4.17 | 168.6 |
| 2 | 4.12 | 169.3 |
| 3 | 4.26 | 168.3 |
| 4 | 4.13 | 167.8 |
| AVG | 4.17 | 168.5 |
| SD | 0.06 | 0.6 |
| % RSD | 1.55 | 0.4 |

AVG: Average
SD: Standard deviation
RSD: Relative standard deviation

### Acceptance Criteria: RSD: ≤2.0%

The peak area RSD from the four consecutive standard solution injections was 0.4%. The acceptance criteria were thus met.

TABLE 2.4
Linearity for Ketamine HCl

| Conc. (μg/mL) | Peak Area |
|---|---|
| 0.300 | 34.0 |
| 0.600 | 67.5 |
| 0.900 | 102.0 |
| 1.200 | 134.5 |
| 1.500 | 168.6 |

### Acceptance Criteria: R² Value ≥0.995

The R² value for the five concentration injections was 0.9999. The acceptance criteria were thus met.

### Conclusion:

As can be seen from the above Tables 2.1-2.4, the HPLC method passed the acceptance criteria of precision and linearity. This developed method was used for assay and impurity analysis of lidocaine and ketamine HCl. This method could detect concentration of 0.3 ug/mL of each lidocaine and ketamine HCl in the drug product.

### Example 2

### Preparation of Drug Delivery Matrix System

The drug delivery matrix system was prepared using the composition as per Table 3.

TABLE 3
Composition of drug delivery matrix system

| Component | (% w/w) | (g/200 g) |
|---|---|---|
| Methacrylic Acid-Ethyl | 16.00 | 32.0 |
| Acrylate Copolymer (1:1) | | |
| Triethanolamine | 1.20 | 2.4 |
| Isopropyl myristate | 5.00 | 10.0 |
| Glycerol | 5.00 | 10.0 |
| Triethyl citrate | 4.20 | 8.4 |
| Alcohol anhydrous | 50.00 | 100.00 |
| Distilled Water | QS | QS |

### Procedure:

Following procedure was followed to prepare the drug delivery matrix system.
1. Tare wt. of a 250 mL glass container was recorded.
2. All components as per Table 3 were added into the container.
3. All the components were mixed well to allow all solid to completely dissolve to, using optionally high shear.
4. The semi-transparent drug delivery matrix system thus obtained was stored at room temperature.

### Example 3

### Determination of Solubility and Stability of Lidocaine and Ketamine HCl in a Drug Delivery Matrix System

Solubility of lidocaine and ketamine HCl in drug delivery matrix system and the stability of the drug delivery matrix was evaluated for further incorporation of additional excipients required to meet the requirements for lidocaine and ketamine HCl topical formulation. Lidocaine and ketamine HCl loaded drug delivery matrix was characterized by measuring appearance, precipitation, and lidocaine and ketamine HCL wt. percentage.

### Preparation of Lidocaine and Ketamine HCl Loaded Drug Delivery Matrix:

Drug delivery matrix system was loaded with lidocaine and ketamine HCl as per the following procedure:
1. An empty 20 mL vial was weighed.
2. 250 mg of lidocaine was added into the vial.
3. 4.25 g of drug delivery matrix system was added into the vial.
4. Both the components were mixed well by sonication.
5. 500 mg of ketamine HCl was added into the vial.
6. Components were mixed well by sonication.
7. Formation of any precipitation was observed after 24 hours. Appearance of lidocaine and ketamine HCl drug delivery matrix system and the solubility and stability of the drug delivery matrix was determined.

TABLE 4
Solubility and stability of lidocaine and
ketamine HCl in drug delivery matrix

| | Ketamine | Precipitation | |
|---|---|---|---|
| Lidocaine (%) | HCl (%) | after 24 hours | Stability |
| 5 | 10 | No | 2 days |
| 6 | 12 | No | 4 days |
| 7 | 14 | No | 6 days |

The drug delivery matrix could dissolve 7% lidocaine and 14% ketamine HCl without precipitation. Furthermore, the drug delivery matrix loaded with lidocaine and ketamine HCl was stable for six or more days.

### Example 4

### Preparation of Topical Formulations

Topical formulations comprising lidocaine and ketamine HCl, drug delivery matrix system, and skin penetration enhancers were prepared using the composition as per Tables 5 and 7, and by compounding the components as per Tables 6 and 8.

TABLE 5
Composition (% w/w)

| Component | F1 | F2 | F3 | F4 | F5 | F6 |
|---|---|---|---|---|---|---|
| Lidocaine | 5 | 5 | 5 | 5 | 5 | 5 |
| Ketamine HCl | 10 | 10 | 10 | 10 | 10 | 10 |
| Limonene | | 10 | | | | |
| Ethyl Oleate | | | 0.2 | | | |
| 1,8-cineole | | | | 5 | | |
| L-menthol | | | | | 5 | |
| Linalool | | | | | | 5 |
| Drug delivery | 85 | 75 | 84.8 | 80 | 80 | 80 |
| matrix system | | | | | | |
| Total | 100 | 100 | 100 | 100 | 100 | 100 |

TABLE 6
Compounding (mg/2 g batch)

| Component | F1 | F2 | F3 | F4 | F5 | F6 |
|---|---|---|---|---|---|---|
| Lidocaine | 100 | 100 | 100 | 100 | 100 | 100 |
| Ketamine HCl | 200 | 200 | 200 | 200 | 200 | 200 |
| Limonene | | 200 | | | | |
| Ethyl Oleate | | | 4 | | | |
| 1,8-cineole | | | | 100 | | |
| L-menthol | | | | | 100 | |
| Linalool | | | | | | 100 |
| Drug delivery | 1700 | 1500 | 1696 | 1600 | 1600 | 1600 |
| matrix system | | | | | | |
| Total | 2000 | 2000 | 2000 | 2000 | 2000 | 2000 |

TABLE 7
Composition (% w/w)

| Component | F7 | F8 | F9 | F10 | F11 | F12 |
|---|---|---|---|---|---|---|
| Lidocaine | 5 | 5 | 5 | 5 | 5 | 5 |
| Ketamine HCl | 10 | 10 | 10 | 10 | 10 | 10 |
| Ethyl Oleate | 2 | | | | | |
| Diethylene glycol | | 10 | | | | |
| monoethyl ether | | | | | | |
| (Transcutol HP) | | | | | | |
| Polyoxyethylene 20 | | | 5 | | | |
| sorbitan monolaurate | | | | | | |
| (Tween 20) | | | | | | |
| DMSO | | | | 10 | | |
| Oleic Acid | | | | | 10 | |
| Polyoxyethylene 20 | | | | | | 5 |
| sorbitan monooleate | | | | | | |
| (Tween 80) | | | | | | |
| Drug delivery | 83 | 75 | 80 | 75 | 75 | 80 |
| matrix system | | | | | | |
| Total | 100 | 100 | 100 | 100 | 100 | 100 |

TABLE 8
Compounding (mg/2 g or 6 g)

| Component | F7* | F8 | F9 | F10 | F11 | F12 |
|---|---|---|---|---|---|---|
| Lidocaine | 300 | 100 | 100 | 100 | 100 | 5 |
| Ketamine HCl | 600 | 200 | 200 | 200 | 200 | 10 |
| Ethyl Oleate | 120 | | | | | |
| Diethylene glycol | | 200 | | | | |
| monoethyl ether | | | | | | |
| (Transcutol HP) | | | | | | |
| Polyoxyethylene 20 | | | 100 | | | |
| sorbitan monolaurate | | | | | | |
| (Tween 20) | | | | | | |
| Dimethyl sulfoxide (DMSO) | | | | 200 | | |
| Oleic Acid | | | | | 200 | |
| Polyoxyethylene 20 | | | | | | 100 |
| sorbitan monooleate | | | | | | |
| (Tween 80) | | | | | | |
| Drug delivery | 4980 | 1500 | 1600 | 1500 | 1500 | 1600 |
| matrix system | | | | | | |
| Total | 6000 | 2000 | 2000 | 2000 | 2000 | 2000 |

*6 g batch

### Procedure: Formulation Preparation:

A glass vial was taken and materials as per compounding Tables 6 and 8 were added into it for each formulation. The materials were mixed well to obtain the topical formulation.

### Example 5

In Vitro Transdermal Test for Evaluation of Permeability of the Topical Formulation into Skin

Following procedure was followed for the in vitro skin tests:

### A. Skin Preparation

Processed porcine abdomen skin was cut to size for the Franz diffusion chambers using Dermatome.

### B. In Vitro Transdermal Test

Phosphate buffer saline (PBS) was used in both donor and receiving chambers of the Franz diffusion apparatus to hydrate the skin for 4 hours. PBS was removed from donor chamber and replaced with 1 mL of each of the topical formulation F1-F12 separately. The donor chamber was kept open to allow evaporation of ethanol and make a thin layer on skin. 1 mL sample was collected from the receiving chamber at 0, 1, 3, 6 and 24 h and the collected sample was filtered using 35 μm filters. Each filtered sample was separately injected into HPLC apparatus to determine the % of lidocaine and ketamine HCl permeation through skin.

### C. Results

TABLE 9
% release of lidocaine from F1-F12 at 24 h

| % API release | 1 h | 3 h | 6 h | 24 h |
|---|---|---|---|---|
| F1 | 0.17 | 2.30 | 3.62 | 6.00 |
| F2 | 0.17 | 1.29 | 3.07 | 5.30 |
| F3 | 0.20 | 2.30 | 3.35 | 6.27 |
| F4 | 0.23 | 1.82 | 4.30 | 5.69 |
| F5 | 0.01 | 0.56 | 2.72 | 4.39 |
| F6 | 0.29 | 1.29 | 3.55 | 5.21 |
| F7 | 0.36 | 2.48 | 8.39 | 21.70 |
| F8 | 0.34 | 2.96 | 7.89 | 21.73 |
| F9 | 0.48 | 1.96 | 10.14 | 22.86 |
| F10 | 0.06 | 0.32 | 6.76 | 21.34 |
| F11 | 0.05 | 2.08 | 7.20 | 17.81 |
| F12 | 0.02 | 0.16 | 7.23 | 17.14 |

TABLE 10
% release of ketamine HCl from F1-F12 in 24 hrs

| % API release | 1 hr | 3 hrs | 6 hrs | 24 hrs |
|---|---|---|---|---|
| F1 | 0.11 | 2.00 | 2.82 | 3.98 |
| F2 | 0.06 | 0.48 | 1.40 | 2.10 |
| F3 | 0.14 | 1.89 | 2.18 | 3.67 |
| F4 | 0.17 | 1.40 | 3.09 | 3.37 |
| F5 | 0.01 | 0.35 | 1.40 | 2.62 |
| F6 | 0.21 | 1.33 | 2.67 | 3.22 |
| F7 | 0.28 | 1.93 | 6.36 | 16.19 |
| F8 | 0.32 | 2.22 | 6.07 | 16.48 |
| F9 | 0.40 | 1.54 | 7.90 | 17.42 |
| F10 | 0.04 | 0.29 | 5.19 | 17.27 |
| F11 | 0.04 | 1.44 | 5.34 | 13.70 |
| F12 | 0.03 | 0.14 | 5.88 | 14.48 |

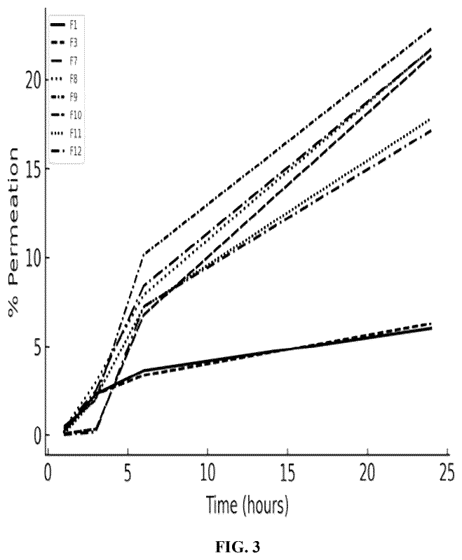

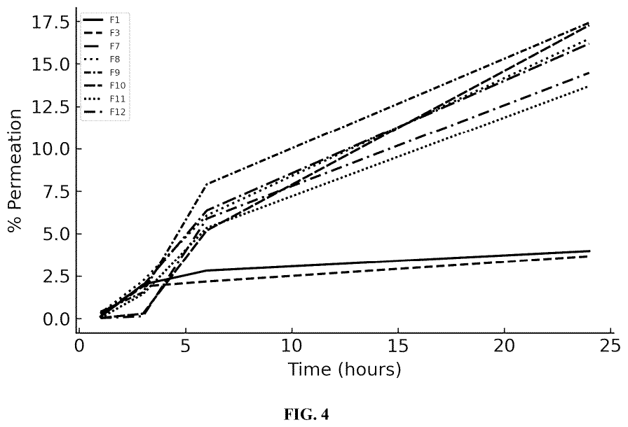

Tables 9-10 show the % release of lidocaine and ketamine HCL from the topical formulations F1-F12 and FIGS. 3 and 4 show the % of lidocaine and ketamine HCl permeated in the skin post application of formulations F1-F12. The formulation F1 had a skin penetration rate of 6% for lidocaine and 3.98% for ketamine after 24 hours. F3, which contained 0.2% ethyl oleate, showed slightly higher penetration for lidocaine at 6.27%. At 24 hours, F9, which contained 5% tween 20, showed a highest skin penetration rate of 22.86% for lidocaine and 17.42% for ketamine HCl compared to other formulations. On the other hand, F8, which contained 10% Diethylene glycol monoethyl ether (Transcutol), had a skin penetration rate of 21.73% for lidocaine and 16.48% for ketamine HCl, and also showed a higher amount of lidocaine (23.55%) and ketamine HCl (22.49%) permeated in the skin.

### Example 6

### Stability Test

Stability studies of formulations F8 and F9 were carried out using the materials as per Table 11.

TABLE 11
Materials

| Sample | Vendor | Grade | Lot |
|---|---|---|---|
| F8 | In House | NA | 485-1-21 |
| F9 | In House | NA | 485-1-21 |

Procedure: Testing were performed for appearance, pH, and HPLC assay of the formulation F8 and F9 of the present invention. For each sample, aliquot 2 g was added into 12 glass vials. Each were labeled accordingly with their respective storage temperature (−20° C., 2-8° C., 25° C., and 40° C.) and time points (1 week, 2 weeks, and 4 weeks). Stability studies results are records in the following Tables 12-15, wherein TO denotes initial, T1/T−1w denotes one-week stability, T2/T−2w denotes two-week stability, and T4/T−4w denotes four-week stability.

TABLE 12
Appearance of F8 and F9

| Name | Storage (° C.) | T0 | T-1 w | T-2 w | T-4 w |
|---|---|---|---|---|---|
| F8 | −20 | Transparent | Transparent | Transparent | Transparent |
| | | gel solution | gel solution | gel solution | gel solution |
| | 2-8 | | Transparent | Transparent | Transparent |
| | | | gel solution | gel solution | gel solution |
| | 25 | | Transparent | Light | Light |
| | | | gel solution | yellowish | yellowish |
| | | | | Transparent | Transparent |
| | | | | gel solution | gel solution |
| | 40 | | Light | Light | Light |
| | | | yellowish | yellowish | yellowish |
| | | | Transparent | Transparent | Transparent |
| | | | gel solution | gel solution | gel solution |
| F9 | −20 | Transparent | Transparent | Transparent | Transparent |
| | | gel solution | gel solution | gel solution | gel solution |
| | 2-8 | | Transparent | Transparent | Transparent |
| | | | gel solution | gel solution | gel solution |
| | 25 | | Transparent | Transparent | Transparent |
| | | | gel solution | gel solution | gel solution |
| | 40 | | Transparent | Transparent | Transparent |
| | | | gel solution | gel solution | gel solution |

TABLE 13
Stability results pH of F8 and F9

| Name | Storage (° C.) | T0 | T-1 w | T-2 w | T-4 w |
|---|---|---|---|---|---|
| F8 | −20 | 6.29 | 6.31 | 6.15 | 6.21 |
| | 2-8 | | 6.29 | 6.14 | 6.22 |
| | 25 | | 6.34 | 6.16 | 6.16 |
| | 40 | | 6.31 | 6.12 | 6.16 |
| F9 | −20 | 6.30 | 6.30 | 6.21 | 6.17 |
| | 2-8 | | 6.34 | 6.15 | 6.15 |
| | 25 | | 6.27 | 6.17 | 6.12 |
| | 40 | | 6.32 | 6.19 | 6.19 |

TABLE 14
Stability Assay (% LC) of F8 and F9

| | | T0 (% LC) | | | | | | | |
|---|---|---|---|---|---|---|---|---|---|
| | Storage | (Recovery) | | T-1 w(LC) | | T-2 w(LC) | | T-4 w(LC) | |
| Name | (° C.) | Lidocaine | Ketamine | Lidocaine | Ketamine | Lidocaine | Ketamine | Lidocaine | Ketamine |
| F8 | −20 | 102.38 | 100.10 | 104.09 | 104.96 | 98.89 | 100.63 | 100.78 | 99.79 |
| | 2-8 | | | 99.83 | 106.40 | 99.41 | 100.70 | 104.20 | 102.07 |
| | 25 | | | 97.57 | 100.68 | 97.93 | 98.57 | 100.42 | 99.22 |
| | 40 | | | 97.52 | 103.53 | 98.01 | 99.12 | 101.18 | 97.48 |
| F9 | −20 | 103.57 | 99.95 | 96.30 | 101.15 | 100.24 | 100.06 | 105.17 | 101.52 |
| | 2-8 | | | 98.17 | 105.08 | 101.39 | 101.43 | 103.60 | 102.36 |
| | 25 | | | 93.95 | 100.31 | 101.14 | 101.29 | 103.59 | 101.99 |
| | 40 | | | 103.84 | 107.65 | 102.30 | 102.08 | 102.74 | 101.07 |

*LC = Label claim (90-110%)

TABLE 15
Impurities of F8 and F9

| | Storage | T0 (% LC) Recovery | | T-1 w(LC) | | T-2 w(LC) | | T-4 w(LC) | |
|---|---|---|---|---|---|---|---|---|---|
| Name | (° C.) | Lidocaine | Ketamine | Lidocaine | Ketamine | Lidocaine | Ketamine | Lidocaine | Ketamine |
| F8 | −20 | None | None | None | None | None | None | None | None |
| | 2-8 | | | None | None | None | None | None | None |
| | 25 | | | None | None | None | None | None | None |
| | 40 | | | None | None | None | None | None | None |
| F9 | −20 | None | None | None | None | None | None | None | None |
| | 2-8 | | | None | None | None | None | None | None |
| | 25 | | | None | None | None | None | None | None |
| | 40 | | | None | None | None | None | None | None |

In all temperature conditions, both formulations F8 and F9 were transparent gel solutions for four weeks except slight variation of F8 at 25° C. (2 and 4 weeks) and F8 40° C. Both formulations' pH was the same at all temperatures—there was no change after four weeks. All formulations showed no sign of active pharmaceutical ingredient (API) degradation based on the HPLC assay results at all temperatures after four weeks.

The foregoing examples are merely illustrative and are not to be taken as limitations upon the scope of the invention. Various changes and modifications to the disclosed embodiments will be apparent to those skilled in the art. Such changes and modifications may be made without departing from the scope of the invention.

### Advantages of the Invention

The present invention provides the topical formulations that dries on the skin and provides a clear film on the skin.

The present invention provides the topical formulation that can be effectively adsorbed through the skin.

The present invention provides the topical formulation that is transparent thereby avoids people being able to see that the patients have applied any pharmaceutical product unlike conventional plasters and patches.

The excipients of the topical formulation of the present invention are water insoluble so that when applied, the topical formulation can sustain getting wet within reason and it does not wash away with water or sweat.

What is claimed is:

1. A topical formulation comprising:
a) one or more active pharmaceutical agents selected from a group comprising of local anesthetic agent and analgesic agent, or pharmaceutically acceptable salts thereof;
b) one or more skin penetration enhancers; and
c) a drug delivery matrix system;
wherein the drug delivery matrix system comprises combination of acrylic based copolymer, alcoholamine, fatty acid ester, polyol, tricarboxylic acid ester, alcohol and solvent.

2. The topical formulation of claim 1, wherein the one or more active pharmaceutical agents comprises at least two pharmaceutical agents.

3. The topical formulation of claim 1, wherein
the local anesthetic agent is selected from lidocaine, bupivacaine, mepivacaine, procaine, tetracaine, and articaine; and
the analgesic agent is selected from ketamine, diclofenac, ketoprofen, ibuprofen, piroxicam, amitriptyline and doxepin.

4. The topical formulation of claim 1, wherein the local anesthetic agent is lidocaine, and the analgesic agent is ketamine.

5. The topical formulation of claim 1, wherein the one or more skin penetration enhancers are selected from selected from the group comprising of diethylene glycol monoethyl ether, polyoxyethylene 20 sorbitan monolaurate, ethyl oleate, 1,8-cineole, linalool, limonene, L-menthol, dimethyl sulfoxide (DMSO), oleic acid, polyoxyethylene 20 sorbitan monooleate, camphor, farnesol, eugenol, geraniol, α-pinene oxide, tetrahydrogeraniol, cymene, and sodium glycolate.

6. The topical formulation of claim 1, wherein
the acrylic based copolymer is selected from polymethacrylate based copolymer, methacrylic acid-ethyl acrylate copolymer, methacrylic acid-methyl methacrylate copolymer, ammonio methacrylate copolymer type A, ammonio methacrylate copolymer type B, acrylates/ethylhexyl acrylate copolymer, and sodium polyacrylate;
the alcoholamine is selected from ethanolamine, diethanolamine, triethanolamine, isopropanolamine, aminomethyl propanol, and 2-amino-2-methyl-1-propanol;
the fatty acid ester is selected from isopropyl myristate, isopropyl palmitate, ethyl oleate, butyl stearate, cetyl palmitate, octyl stearate, lauryl lactate, and oleyl oleate;
the polyol is selected from glycerol, propylene glycol, sorbitol, mannitol, xylitol, butylene glycol, and polyethylene glycol;
the tricarboxylic acid ester is selected from triethyl citrate, tributyl citrate, acetyl triethyl citrate, acetyl tributyl citrate, trimethyl citrate, and triacetin;
the alcohol is selected from anhydrous ethanol, isopropanol, n-propanol, benzyl alcohol, butanol, and denatured alcohol; and
the solvent is distilled water.

7. The topical formulation of claim 1, wherein the drug delivery matrix system comprises combination of methacrylic acid-ethyl acrylate copolymer, triethanolamine, isopropyl myristate, glycerol, triethyl citrate, alcohol anhydrous, and distilled water.

8. The topical formulation of claim 6, wherein
(a) the acrylic based copolymer is present in an amount of about 10% to about 20% by weight of the drug delivery matrix system;
(b) the alcoholamine is present in an amount of about 1% to about 2% by weight of the drug delivery matrix system;
(c) the fatty acid ester is present in an amount of about 3% to about 8% by weight of the drug delivery matrix system;
(d) the polyol is present in an amount of about 3% to about 8% by weight of the drug delivery matrix system;
(e) the tricarboxylic acid ester is present in an amount of about 2% to about 6% by weight of the drug delivery matrix system;
(f) the alcohol is present in an amount of about 30% to about 80% by weight of the drug delivery matrix system; and/or
(g) the solvent is present in quantity sufficient.

9. The topical formulation of claim 1, wherein the one or more active pharmaceutical agents are present in the topical formulation in an amount of about 1% to about 50% by weight.

10. The topical formulation of claim 1, wherein the skin penetration enhancer is present in the topical formulation in an amount of about 0.1% to about 50% by weight.

11. The topical formulation of claim 1, wherein the drug delivery matrix system is present in the topical formulation in an amount of about 60% to about 98.9% by weight.

12. The topical formulation of claim 1, wherein
(a) the anesthetic agent or pharmaceutically acceptable salts thereof is present in an amount of about 2% to about 10% by weight;
(b) the analgesic agent or pharmaceutically acceptable salts thereof is present in an amount of about 5% to about 15% by weight;
(c) the one or more skin penetration enhancers is in an amount of about 0.2% to about 15% by weight; and
(d) the drug delivery matrix system is in an amount of about 70% to about 85% by weight.

13. The topical formulation of claim 1, wherein the formulation optionally comprises a pharmaceutically acceptable excipients selected from buffer, chelating agent, fragrance, preservative, emulsifier, suspending agent, and thickening agent.

14. The topical formulation of claim 1, wherein the formulation is a sustained release formulation.

15. A method of treating or relieving pain comprising administering an effective amount of the topical formulation as claimed in claim 1 to a mammal in need thereof, wherein the administration is done by applying the topical formulation to a site having pain; and wherein the formulation releases the one or more active pharmaceutical agents, or pharmaceutically acceptable salts thereof for 24 hours or more after administration of the formulation to the site.

16. The method of claim 15, wherein the pain is selected from body pain, joint pain, pain caused by injury, pain of region where a needle is injected into the skin, and chronic pain.

17. The method of claim 16, wherein the pain caused by injury comprises wounds or burns.

18. The method of claim 16, wherein the chronic pain is selected from post-herpetic neuralgia, complex regional pain syndrome, and arthritis.

19. The method of claim 15, wherein the pain is a complex regional pain syndrome and the topical formulation first allows local anesthetic agent to numb the extremity along with analgesic agent to allow the patient to undergo physical therapy and continue to provide desensitization to help into remission of pain.
